(19) United States
(12) Patent Application Publication
(10) Pub. No.: US 2023/0187051 A1
(43) Pub. Date: Jun. 15, 2023

(54) SYSTEM AND METHOD FOR PLANNING AND ACQUISITION OF BALANCED PROVISIONS FOR WILDERNESS TRAVEL AND ACTIVITIES

(71) Applicant: RightOnTrek Inc., Kalispell, MT (US)

(72) Inventors: Victoria LIVSCHITZ, Whitefish, MT (US); Anastasia VORONKOVA, Big Sky, MT (US); Eric BOXER, Whitefish, MT (US); Thomas DANGERFIELD, Crestwood, KY (US)

(21) Appl. No.: 17/551,667

(22) Filed: Dec. 15, 2021

(51) Int. Cl. G16H 20/60; G06Q 30/06

(52) U.S. Cl. CPC G16H 20/60; G06Q 30/0621; G06Q 30/0631

(57) ABSTRACT

A computer implemented method in a data processing system comprising a processor and a memory, comprising instructions which are executed by the processor to cause the processor to implement a meal plan optimization for hiking or trekking trips, comprising: receiving a request to generate a meal plan for an individual in a specific trek, said individual identified by at least one of: allergenic tolerances, fitness level, weight, dietary choices, age; said specific trek or hike is identified by at least one of: number of days, level of activity, expected weather; said method calculates the expected number of calories and nutritional contents needed for individual based on all known data; said method calculates a selected meal plan, including varieties, for the individual, and presents said plan to the individual.

### FIELD OF INVENTION

The present disclosure relates to hike or trek preparation, and more specifically to an automated system for planning needed provisions for specific planned treks or hikes.

### BACKGROUND OF INVENTION

Awareness to healthy and nutritious food is always around, with research being conducted without stop and recommendations changing constantly. Individual food suggestions are quickly taking the front seat, as each of us has different requirements, and different likes and dislikes. In similar fashion, allergenics and dietary choices are quickly taking more importance in meal selections. There is more awareness today for food components that can cause damage when consumed by an allergic individual. The same is true for dietary choices such as vegans and vegetarians who will only eat certain foods with certain components in them.

For this reason, meal preparation for an individual has become complicated. This preparation complexity doubles when a meal plan is desired for a group of people with different requirements, allergenics and dietary choices. The preparation complexity compounds even further when said meal plans are to be decided in advance for specific hiking trips. Such trips can span several days, for a group of people of different ages and fitness levels, for hiking trips that involve varying degrees of difficulty levels, all while allowing for variations in the menu so participants will not have to eat the same food repeatedly on all days of the trip. All of this, while also providing each hiker with all needed nutrients and calorie intake needed to match the level of activity expected during, as well as the weather, etc.

The complexity of group food planning is further compounded in the wilderness. Groups mostly prepare their food together with shared equipment—the problem there arises when different group members have different preferences, and more importantly, different allergies—which can be especially difficult when cooking the meals in the same equipment. Such preparations have to be planned in advance, as these expeditions are far from any option to restock and need to take in all of the equipment with them. This means that everything carried by such expeditions needs to be preplanned accurately.

In view of the above, there is still an unmet long-felt need for a system that will automatically generate a meal plan for individuals and groups, including variations, based on the travel expedition, individual health, dietary choices, and nutrition values, restrictions as well as user preferences.

### SUMMARY OF THE INVENTION

It is thus one object of the present invention to describe a computer implemented method in a data processing system comprising a processor and a memory, comprising instructions which are executed by the processor to cause the processor to implement a meal plan optimization for hiking or trekking trips, comprising: receiving a request to generate a meal plan for an individual in a specific trek, said individual identified by at least one of: allergenic tolerances, fitness level, weight, dietary choices, age; said specific trek or hike is identified by at least one of: number of days, level of activity, expected weather; said method calculates the expected number of calories and nutritional contents needed for individual based on all known data; said method calculates a selected meal plan, including varieties, for the individual, and presents said plan to the individual.

It is another object of the present invention to describe a method that create a meal plan for a group of people, separately identifying requirements for each individual in the group.

It is another object of the present invention to describe a method wherein the meals suggested to an individual are based on a list of pre-prepared meals.

It is another object of the present invention to describe a method wherein an individual can manipulate suggested meal plan according to his own taste.

It is another object of the present invention to describe a method wherein pre-packed meals in said meal plan can be ordered from an online store.

It is yet another object of the present invention to describe a system for generating an optimized meal plan for hiking or trekking trips, comprising:

a processor, a memory, wherein: a first module configured to receive a user request the preparation of a meal plan and receive information regarding the trek with at least one of: location, target dates, trek length, planned activities; said module receives information regarding food preferences: dietary choice, allergies, health conditions, weight, fitness level; a second meal analytics module configured to prepare meal suggestions comprising: a meal components and information module used to identify specific foods for their allergenic and nutritional values, a user preferences and information module used to identify individual allergies, caloric needs, a trek information module configured to identify trek length, activity levels and weather information; said meal analytics engine combines data from all modules to create a suggested meal plan for an individual.

It is another object of the present invention to describe the system wherein the system is accessible from all web connected devices, including web browsers, mobile phones and tablet computers.

It is another object of the present invention to describe the system wherein an e-commerce module is added to allow a user to order pre-packed meals based on the given suggestions.

It is another object of the present invention to describe the system wherein the system can calculate meal information based on components and method of preparation.

It is another object of the present invention to describe the system wherein the system described in claim 6, wherein the system can add suggestions of variations of meals for a user to choose from.

### BRIEF DESCRIPTION OF THE FIGURES

The accompanying drawings, which are included to provide a further understanding of the invention and are incorporated in and constitute apart of this specification, illustrate embodiments of the invention and together with the description serve to explain the principles of the invention.

FIG. 1 depicting a schematic presentation of the overall system that provides a user, or a group of users the ability to plan a set of meals based on a specific hiking trip.

FIG. 2 depicting a schematic presentation of the meal analytics engine.

FIG. 3 depicting a schematic presentation of hiking trip data gathering and organization.

FIG. 4 depicting a schematic presentation of user information data gathering and organization.

FIG. 5 depicting a schematic presentation of meal components and nutrition values data gathering and organization.

FIG. 6 depicting a flow chart of a machine learning training model for generating meal plans.

### DETAILED DESCRIPTION OF PREFERRED EMBODIMENTS

The following description is provided, alongside all chapters of the present invention, so as to enable any person skilled in the art to make use of the invention and sets forth the best modes contemplated by the inventor of carrying out this invention. Various modifications, however, are adapted to remain apparent to those skilled in the art, since the generic principles of the present invention have been defined specifically to provide a method of analyzing specific provision requirements for hiking trips and generating a list of specific provisions needed based on individual requirements.

As used herein after, the term “ETL” refers to an Extract, Transform, Load layer for combining data from multiple systems and sources into a single database.

U.S. Pat. No. 10,373,522 Generative group-based meal planning system and method describes a system for generating meal plans based on a pre-selected list of ingredients, specific genetic needs, etc. The patent also describes receiving recipes for said meal plans that are designed for specific people (so-called genetic profiling), costs, preparation time. The system remarks about allergenic foods and nutritional value. This patent defers from the current invention by purpose. The current invention is set to check the specific needs of individuals or groups going on planned hiking trips. As such the nutritional values of the meals must reflect the expected needs specific to a trip, weather conditions, etc. In addition, Hiking trip meals come prepared, and the system allows a user to shop for a variety of prepared meals that will satisfy their exact needs for said trip.

U.S. Pat. No. 10,496,794 systems and methods for providing meal plans concerns a method of characterizing a person's taste based on the purchases said person made in various retail outlets. The system then suggests recipes to the user based on personalized tastes. This differs from the current invention, that plans meals specifically for hiking trips and treks, such food needs to be nutritious, planned of time for several days, provide enough caloric intake based on the activity level and prepacked for the road.

EP patent application No. 3721436 an apparatus and method for personalized meal plan describes a method of acquiring data from a client, generating nutrient values, and creating a meal plan with recipes for the user. This differs from the current invention that provides meal plans for individuals and groups (and an option to buy pre-packed food) for hiking and treks.

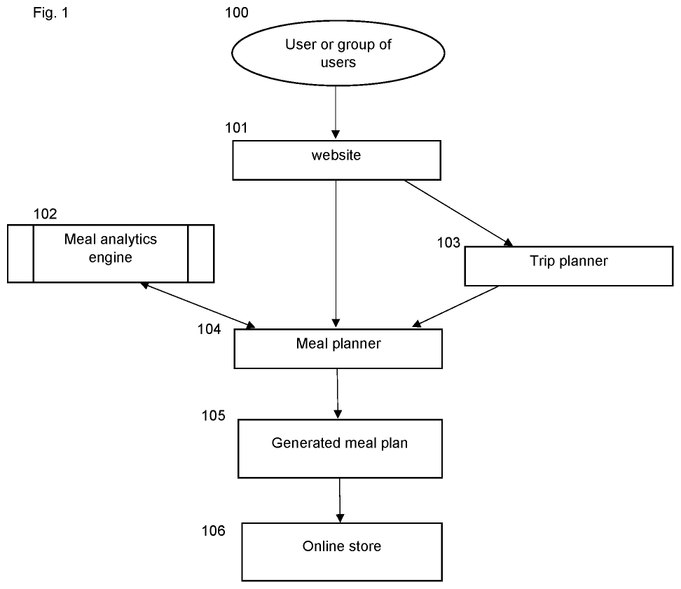

FIG. 1 depicts a high-level view of the meal plan generation system where a user, or a group of users (100) will go into a specific website for preparation for their hiking trip. The user can then decide to use only a meal planner subsystem (104) or use a hiking trip planner (103) which will also user the meal planner to associate provisions with said trip. The meal planner will retain information from a meal analytics engine (102) and generate a specific meal plan, with all factors considered (105). The user can then buy all said meals in an online store (106).

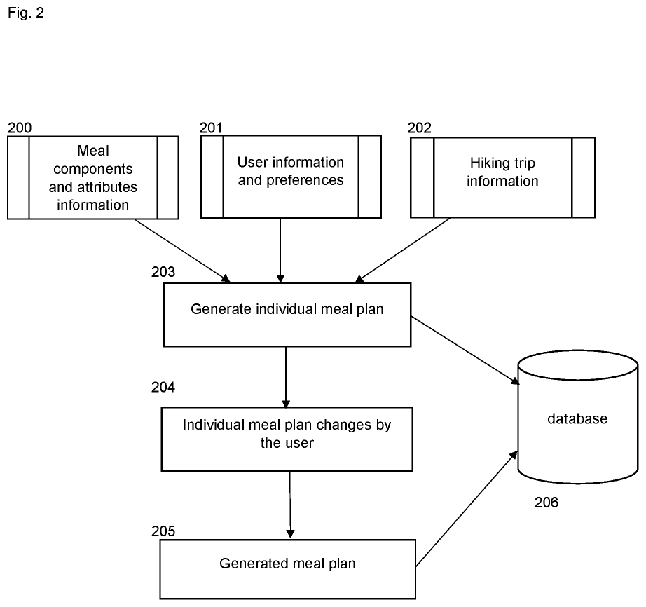

FIG. 2 shows the components of the meal analytics engine (102). The analytics engine can use up to three different data subsystems and a database (206) to help generate a meal plan (203). The system will take into account all relevant information about a specific hiking trip (202), user information, preferences and health conditions (201) as well as available meal components and health attributes (200). The system then allows the user to make changes as he sees fit to the proposed meal plan (204), and generates the complete plan to be used for purchases, etc. (205). The generated meal plan is stored in the database (206) for possible future use and to help the system adjust generated meals for similar hiking trips for other users.

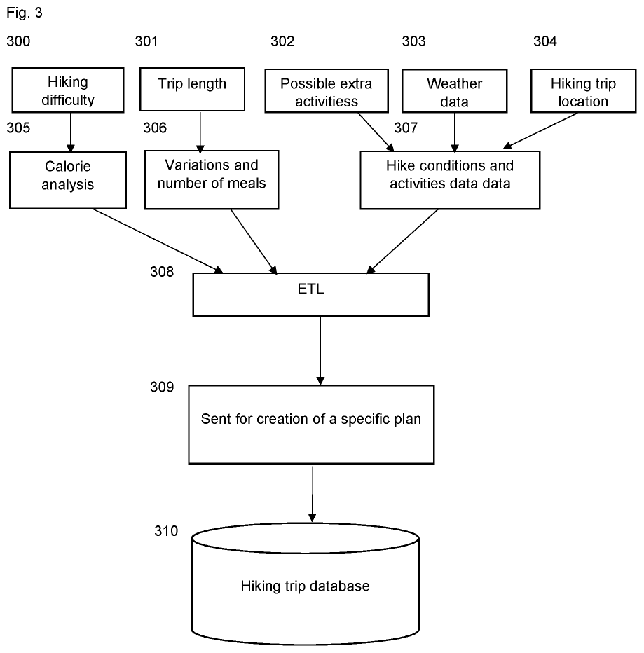

FIG. 3 depicts the collected data for a specific hiking trip for use in the meal generation system. The system checks the hiking path difficulty (300) for calorie intake analysis (305), it checks the wanted trip length (301) for the number of meals needed (including spares) as well as variations of meals, so a user will not get bored eating the same meals all through the hiking trip (306). The system then collects data about the hiking trip location (304), projected weather on the dates on the trips (303) and possible extra activities through the specific trek (canyoning, walking through river streams, etc.) (302). All the latter are combined into the hike conditions and activities data (307). The systems then use an ETL (extract, transform load) layer to combine all data (308) which will first be sent to be used in the creation of a specific meal plan (309) and will be also saved in a specific database (310).

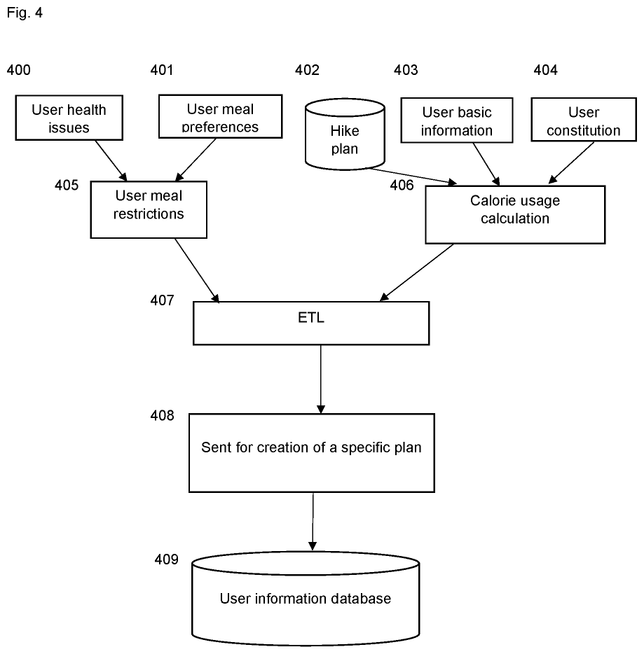

FIG. 4 depicts the collected data for a specific user for use in the meal generation system. The system uses a user's basic information (age, weight, etc.) (403) and the user's constitution level (fitness level) (404), in conjunction with the planned hike trip data (402) to create a calorie usage calculation (406) for the specific intended trip. The system also checks for a user's health issues (400) and meal preferences (vegan, vegetarian, allergies) (401) to create a list of user meal restrictions (405). All the data is sent to an ETL layer, which extracts and transforms the information into a single dataset that is sent to the meal generation system, and also saved in a specific database (409).

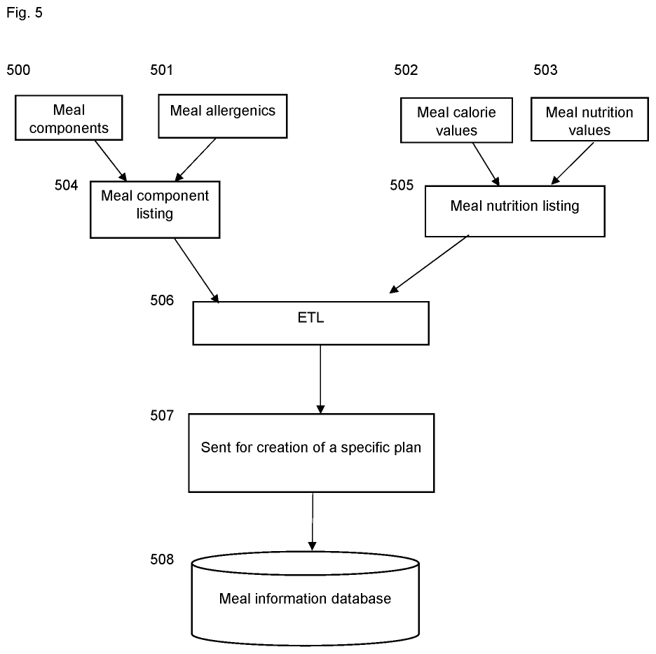

FIG. 5 depicts the collected data for specific meals. Meal components (500) and meal allergenics (501) are both combined into a single meal component listing (504). The same way, meal calorie values (502) and meal nutrition values (503) are combined into a single meal nutrition listing (505). Both values are transferred to an ETL layer (506) which transforms the information into a single dataset and sent to the meal generation system (507), as well to as to a meal database (508).

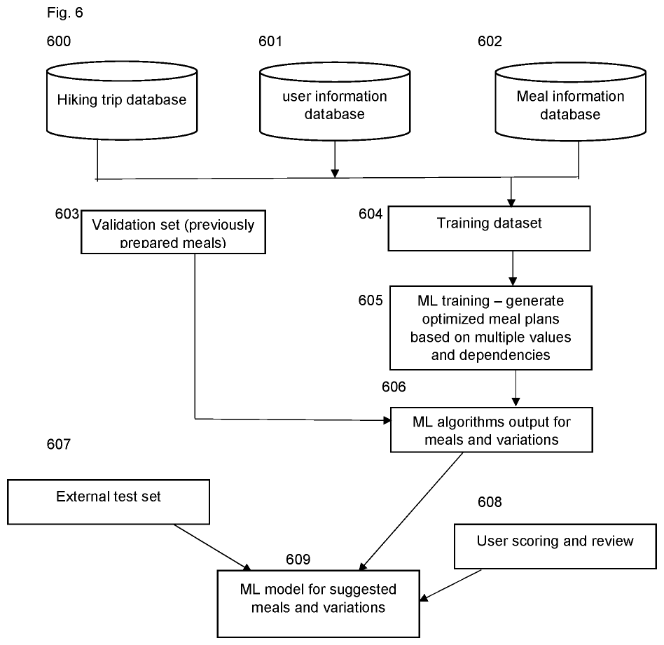

FIG. 6 depicts a machine learning training model. The system is fed information from a hiking trip database (600), user information database (601) and meal information database (602). The data is classified as a training set (604) with which the machine learning module prepares algorithms to calculate meal plans based on all the data (605). The prepared machine learning algorithms (606) are checked a validation set of previously prepared meals (603). The system than creates a machine learning model (609) for suggested meals and variation of meals using the proposed algorithms tested against external test datasets (607). Said model is continuously updated against user reviews of the meal plans (608).

The present invention provides a method of preparing food plans for individuals or groups using a machine learning module to analyze all relevant information about a specific hike or trek (including weather conditions, trek difficulty, etc.), compare said information with an individual's food requirements (calorie intake based on age, fitness level, dietary choices (e.g. veganism), allergies, etc.). From said comparison the system can extrapolate the nutritious values, components, calorie values, etc. of the individual, and prepare a meal plan from a list pre-packed food packets based on these requirements.

In another embodiment, the invention provides a method that can use the preferences of each individual for meals and snacks, including food based on specific habits and desires—like do not suggest food from a specific brand, tea or coffee in the morning for an adult, hot coco for a child, etc. Such preferences can also be incorporated into the prepared meal plan for each individual.

In another embodiment, the present invention provides a method for suggesting the numbers and types of cooking utensils needed in an expedition or trek, based on the meals, allergies, and preferences of each individual in a group. When a group includes a person who is allergic to some ingredient, the group might need to bring along extra cooking utensils to separate the preparation of the food to avoid causing an allergic reaction. In addition, when a vegetarian, or vegan, is part of the group, they might prefer the vegan food be prepared separately from the meat based meals.

### Examples

As an example, a family of five plans a camping and hiking trip. The family comprises of a father and mother in their mid-forties, the oldest daughter at 13, middle son-10 and youngest daughter 7. One of the parents logs into the website and plans the trip. The first part is planning the camping location, and planning activities and hikes in said area. The parent than answers a basic questionnaire on various likes and dislikes of each family member, as well as specific allergies. The system then uses that information, calculates nutritional needs for each group member, based on trip location, expected weather, activity level, variety, nutritional needs, etc. Said meal plan is presented to the parent who can change as they see fit. All information is saved to the system for future reference both for this specific family, as well as for comparison and for determining similar products to other families. The parent can then be directed to buy all meals pre-prepared for all family members.

On another example, a group of friends decide to go on a five-day hike on a trek in Montana. The group enter the website, inputs all relevant data on the trek itself and on their preferences. Said data is used to generate suggestions based on the systems' data sources regarding calorie usage on a specific trek (based on weather conditions, weight, fitness level, etc.). The system generates a plan for each individual member according to their own needs, allergenic profiles, and dietary choices. The system then determines meal compositions (with variety built in) for each member out of a pre-prepared set of meals. The group can then use the system to buy all said meals ahead of time and go out to the trek with all the food they'll need.

Another example can be a hunting group of five going on a guided tour, the group meal plan is prepared based on all the data about the trip (location, date, expected weather, etc.). In addition, in the case of allergies and different preferences, the system can determine the group needs to pack more cooking sets, to make sure allergenics do not cross between the hunting group. Further, the planner will attempt to suggest a common meal plan for everyone involved in the trip, such a plan can reduce the equipment that needs to be carried.

1. A computer implemented method in a data processing system comprising a processor and a memory, comprising instructions which are executed by the processor to cause the processor to implement a meal plan optimization for hiking or trekking trips, comprising:
receiving a request to generate a meal plan for an individual in a specific trek, said individual identified by at least one of: allergenic tolerances, fitness level, weight, dietary choices, age; said specific trek or hike is identified by at least one of: number of days, level of activity, expected weather; said method calculates the expected number of calories and nutritional contents needed for individual based on all known data; said method calculates a selected meal plan, including varieties, for the individual, and presents said plan to the individual.

2. The method described in claim 1, wherein the method can create a meal plan for a group of people, separately identifying requirements for each individual in the group.

3. The method described in claim 1, wherein the meals suggested to an individual are based on a list of pre-prepared meals.

4. The method described in claim 1, wherein an individual can manipulate suggested meal plan according to his own taste.

5. The method described in claim 1, wherein pre-packed meals in said meal plan can be ordered from an online store.

6. A system for generating an optimized meal plan for hiking or trekking trips, comprising:
a processor, a memory, wherein: a first module configured to receive a user request the preparation of a meal plan and receive information regarding the trek with at least one of: location, target dates, trek length, planned activities; said module receives information regarding food preferences: dietary choice, allergies, health conditions, weight, fitness level; a second meal analytics module configured to prepare meal suggestions comprising: a meal components and information module used to identify specific foods for their allergenic and nutritional values, a user preferences and information module used to identify individual allergies, caloric needs, a trek information module configured to identify trek length, activity levels and weather information; said meal analytics engine combines data from all modules to create a suggested meal plan for an individual.

7. The system described in claim 6, wherein the system is accessible from all web connected devices, including web browsers, mobile phones and tablet computers.

8. The system described in claim 6, wherein an e-commerce module is added to allow a user to order pre-packed meals based on the given suggestions.

9. The system described in claim 6, wherein the system can calculate meal information based on components and method of preparation.

10. The system described in claim 6, wherein the system can add suggestions of variations of meals for a user to choose from.
